SEQUENCE LISTING (130 sequences)

SEQ ID NO: 1 - protein, 304 aa, Rhodopseudomonas palustris
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYHWSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 2 - DNA, 912 bp, Rhodopseudomonas palustris
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctaccactggtctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 3 - DNA, 37 bp, Artificial Sequence
  H155W156-Forward primer
  misc_feature (16)..(17): n is a, c, g, or t
  misc_feature (18)..(18): k is g or t
  misc_feature (19)..(20): n is a, c, g, or t
  misc_feature (21)..(21): k is g or t
gcactgaaaatctatnnknnkagctttctggcacagc

SEQ ID NO: 4 - DNA, 37 bp, Artificial Sequence
  H155W156-Reverse primer
  misc_feature (17)..(17): m is a or c
  misc_feature (18)..(19): n is a, c, g, or t
  misc_feature (20)..(20): m is a or c
  misc_feature (21)..(22): n is a, c, g, or t
gctgtgccagaaagctmnnmnnatagattttcagtgc

SEQ ID NO: 5 - DNA, 34 bp, Artificial Sequence
  Y219-Forward primer
  misc_feature (16)..(17): n is a, c, g, or t
  misc_feature (18)..(18): k is g or t
gtgatgtgtgaagatnnkcgtgcgggtgcgtatg

SEQ ID NO: 6 - DNA, 34 bp, Artificial Sequence
  Y219-Reverse primer
  misc_feature (17)..(17): m is a or c
  misc_feature (18)..(19): n is a, c, g, or t
catacgcacccgcacgmnnatcttcacacatcac

SEQ ID NO: 7 - protein, 304 aa, Artificial Sequence
  fluoroacetate dehalogenase mutant1(H155I,W156S,Y219F)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYISSFLAQPAPLPENLLGGDPDFYVKAKLASWTRAGDLSAFDPRAVEHYRIAFADPMRRHVMCEDFRAGAYADFEHDKIDVEAGNKIPVPMLALWGASGIAQSAATPLDVWRKWASDVQGAPIESGHFLPEEAPDQTAEALVRFFSAAPGS

SEQ ID NO: 8 - DNA, 912 bp, Artificial Sequence
  fluoroacetate dehalogenase mutant1(H155I,W156S,Y219F)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacatctcttctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagacttccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 9 - protein, 304 aa, Artificial Sequence
  fluoroacetate dehalogenase mutant2(H155V,W156M,Y219L)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYVMSFLAQPAPLPENLLGGDPDFYVKAKLASWTRAGDLSAFDPRAVEHYRIAFADPMRRHVMCEDLRAGAYADFEHDKIDVEAGNKIPVPMLALWGASGIAQSAATPLDVWRKWASDVQGAPIESGHFLPEEAPDQTAEALVRFFSAAPGS

SEQ ID NO: 10 - DNA, 912 bp, Artificial Sequence
  fluoroacetate dehalogenase mutant2(H155V,W156M,Y219L)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacgttatgtctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagacctgcgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 11 - protein, 304 aa, Artificial Sequence
  fluoroacetate dehalogenase mutant3(H155V,W156R,Y219M)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYVRSFLAQPAPLPENLLGGDPDFYVKAKLASWTRAGDLSAFDPRAVEHYRIAFADPMRRHVMCEDMRAGAYADFEHDKIDVEAGNKIPVPMLALWGASGIAQSAATPLDVWRKWASDVQGAPIESGHFLPEEAPDQTAEALVRFFSAAPGS

SEQ ID NO: 12 - DNA, 912 bp, Artificial Sequence
  fluoroacetate dehalogenase mutant3(H155V,W156R,Y219M)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacgttcgttctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagacatgcgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 13 - protein, 304 aa, Artificial Sequence
  fluoroacetate dehalogenase mutant4(H155I,W156S)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYISSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 14 - DNA, 912 bp, Artificial Sequence
  fluoroacetate dehalogenase mutant4(H155I,W156S)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacatctcttctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 15 - protein, 304 aa, Artificial Sequence
  fluoroacetate dehalogenase mutant5(H155N,W156F)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYNFSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 16 - DNA, 912 bp, Artificial Sequence
  fluoroacetate dehalogenase mutant5(H155N,W156F)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacaacttctctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 17 - protein, 304 aa, Artificial Sequence
  fluoroacetate dehalogenase mutant6(H155V,W156T)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYVTSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 18 - DNA, 912 bp, Artificial Sequence
  fluoroacetate dehalogenase mutant6(H155V,W156T)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacgttacctctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 19 - protein, 304 aa, Artificial Sequence
  fluoroacetate dehalogenase mutant7(W156M)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYHMSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 20 - DNA, 912 bp, Artificial Sequence
  fluoroacetate dehalogenase mutant7(W156M)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctaccacatgtctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 21 - protein, 304 aa, Artificial Sequence
  fluoroacetate dehalogenase mutant8(H155V,W156M)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYVMSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 22 - DNA, 912 bp, Artificial Sequence
  fluoroacetate dehalogenase mutant8(H155V,W156M)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacgttatgtctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 23 - protein, 304 aa, Artificial Sequence
  fluoroacetate dehalogenase mutant9(H155V,W156R)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYVRSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 24 - DNA, 912 bp, Artificial Sequence
  fluoroacetate dehalogenase mutant9(H155V,W156R)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacgttcgttctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 25 - protein, 304 aa, Artificial Sequence
  fluoroacetate dehalogenase mutant10(H155Q,W156F)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYQFSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 26 - DNA, 912 bp, Artificial Sequence
  fluoroacetate dehalogenase mutant10(H155Q,W156F)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctaccagttctctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 27 - protein, 304 aa, Artificial Sequence
  fluoroacetate dehalogenase mutant11(H155L,W156S)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYLSSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 28 - DNA, 912 bp, Artificial Sequence
  fluoroacetate dehalogenase mutant11(H155L,W156S)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacctgtcttctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 29 - protein, 304 aa, Artificial Sequence
  fluoroacetate dehalogenase mutant12(H155F,W156G)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYFGSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 30 - DNA, 912 bp, Artificial Sequence
  fluoroacetate dehalogenase mutant12(H155F,W156G)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacttcggttctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 31 - protein, 304 aa, Artificial Sequence
  fluoroacetate dehalogenase mutant13(H155F,W156L)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYFLSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 32 - DNA, 912 bp, Artificial Sequence
  fluoroacetate dehalogenase mutant13(H155F,W156L)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacttcctgtctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 33 - protein, 304 aa, Artificial Sequence
  fluoroacetate dehalogenase mutant14(H155Q,W156L)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYQLSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 34 - DNA, 912 bp, Artificial Sequence
  fluoroacetate dehalogenase mutant14(H155Q,W156L)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctaccagctgtctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 35 - protein, 304 aa, Artificial Sequence
  fluoroacetate dehalogenase mutant15(H155V,W156D)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYVDSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 36 - DNA, 912 bp, Artificial Sequence
  fluoroacetate dehalogenase mutant15(H155V,W156D)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacgttgactctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 37 - protein, 304 aa, Artificial Sequence
  fluoroacetate dehalogenase mutant16(H155M,W156A)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYMASFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 38 - DNA, 912 bp, Artificial Sequence
  fluoroacetate dehalogenase mutant16(H155M,W156A)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacatggcttctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 39 - protein, 304 aa, Artificial Sequence
  fluoroacetate dehalogenase mutant17(H155L,W156I)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYLISFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 40 - DNA, 912 bp, Artificial Sequence
  fluoroacetate dehalogenase mutant17(H155L,W156I)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacctgatctctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 41 - protein, 304 aa, Artificial Sequence
  fluoroacetate dehalogenase mutant18(H155F,W156M)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYFMSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 42 - DNA, 912 bp, Artificial Sequence
  fluoroacetate dehalogenase mutant18(H155F,W156M)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacttcatgtctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 43 - protein, 304 aa, Artificial Sequence
  fluoroacetate dehalogenase mutant19(H155F,W156R)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYFRSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 44 - DNA, 912 bp, Artificial Sequence
  fluoroacetate dehalogenase mutant19(H155F,W156R)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacttccgttctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 45 - protein, 304 aa, Artificial Sequence
  fluoroacetate dehalogenase mutant20(H155A,W156F)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYAFSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 46 - DNA, 912 bp, Artificial Sequence
  fluoroacetate dehalogenase mutant20(H155A,W156F)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacgctttctctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 47 - protein, 304 aa, Artificial Sequence
  fluoroacetate dehalogenase mutant21(H155P,W156Y)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYPYSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 48 - DNA, 912 bp, Artificial Sequence
  fluoroacetate dehalogenase mutant21(H155P,W156Y)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacccgtactctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 49 - protein, 304 aa, Artificial Sequence
  fluoroacetate dehalogenase mutant22(W156T)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYHTSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 50 - DNA, 912 bp, Artificial Sequence
  fluoroacetate dehalogenase mutant22(W156T)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctaccacacctctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 51 - protein, 304 aa, Artificial Sequence
  fluoroacetate dehalogenase mutant23(H155V)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYVWSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 52 - DNA, 912 bp, Artificial Sequence
  fluoroacetate dehalogenase mutant23(H155V)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacgtttggtctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 53 - protein, 304 aa, Artificial Sequence
  fluoroacetate dehalogenase mutant24(W156C)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYHCSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 54 - DNA, 912 bp, Artificial Sequence
  fluoroacetate dehalogenase mutant24(W156C)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctaccactgctctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 55 - protein, 304 aa, Artificial Sequence
  fluoroacetate dehalogenase mutant25(H155T,W156F)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYTFSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 56 - DNA, 912 bp, Artificial Sequence
  fluoroacetate dehalogenase mutant25(H155T,W156F)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacaccttctctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 57 - protein, 304 aa, Artificial Sequence
  fluoroacetate dehalogenase mutant26(H155W,W156I)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYWISFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 58 - DNA, 912 bp, Artificial Sequence
  fluoroacetate dehalogenase mutant26(H155W,W156I)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctactggatctctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 59 - protein, 304 aa, Artificial Sequence
  fluoroacetate dehalogenase mutant27(H155L,W156L)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYLLSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 60 - DNA, 912 bp, Artificial Sequence
  fluoroacetate dehalogenase mutant27(H155L,W156L)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacctgctgtctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 61 - protein, 304 aa, Artificial Sequence
  fluoroacetate dehalogenase mutant28(H155P,W156T)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYPTSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 62 - DNA, 912 bp, Artificial Sequence
  fluoroacetate dehalogenase mutant28(H155P,W156T)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacccgacctctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 63 - protein, 304 aa, Artificial Sequence
  fluoroacetate dehalogenase mutant29(H155C,W156M)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYCMSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 64 - DNA, 912 bp, Artificial Sequence
  fluoroacetate dehalogenase mutant29(H155C,W156M)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctactgcatgtctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 65 - protein, 304 aa, Artificial Sequence
  fluoroacetate dehalogenase mutant30(H155I,W156A)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYIASFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 66 - DNA, 912 bp, Artificial Sequence
  fluoroacetate dehalogenase mutant30(H155I,W156A)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacatcgcttctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 67 - protein, 304 aa, Artificial Sequence
  fluoroacetate dehalogenase mutant31(H155L,W156A)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYLASFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 68 - DNA, 912 bp, Artificial Sequence
  fluoroacetate dehalogenase mutant31(H155L,W156A)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacctggcttctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 69 - protein, 304 aa, Artificial Sequence
  fluoroacetate dehalogenase mutant32(W156F)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYHFSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 70 - DNA, 912 bp, Artificial Sequence
  fluoroacetate dehalogenase mutant32(W156F)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctaccacttctctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 71 - protein, 304 aa, Artificial Sequence
  fluoroacetate dehalogenase mutant33(H155T,W156L)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYTLSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 72 - DNA, 912 bp, Artificial Sequence
  fluoroacetate dehalogenase mutant33(H155T,W156L)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacaccctgtctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 73 - protein, 304 aa, Artificial Sequence
  fluoroacetate dehalogenase mutant34(H155V,W156Y)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYVYSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 74 - DNA, 912 bp, Artificial Sequence
  fluoroacetate dehalogenase mutant34(H155V,W156Y)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacgtttactctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 75 - protein, 304 aa, Artificial Sequence
  fluoroacetate dehalogenase mutant35(H155I,W156P)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYIPSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 76 - DNA, 912 bp, Artificial Sequence
  fluoroacetate dehalogenase mutant35(H155I,W156P)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacatcccgtctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 77 - protein, 304 aa, Artificial Sequence
  fluoroacetate dehalogenase mutant36(H155L,W156F)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYLFSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 78 - DNA, 912 bp, Artificial Sequence
  fluoroacetate dehalogenase mutant36(H155L,W156F)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacctgttctctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 79 - protein, 304 aa, Artificial Sequence
  fluoroacetate dehalogenase mutant37(H155V,W156G)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYVGSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 80 - DNA, 912 bp, Artificial Sequence
  fluoroacetate dehalogenase mutant37(H155V,W156G)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacgttggttctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 81 - protein, 304 aa, Artificial Sequence
  fluoroacetate dehalogenase mutant38(H155F,W156T)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYFTSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 82 - DNA, 912 bp, Artificial Sequence
  fluoroacetate dehalogenase mutant38(H155F,W156T)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacttcacctctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 83 - protein, 304 aa, Artificial Sequence
  fluoroacetate dehalogenase mutant39(H155A,W156M)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYAMSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 84 - DNA, 912 bp, Artificial Sequence
  fluoroacetate dehalogenase mutant39(H155A,W156M)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacgctatgtctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 85 - protein, 304 aa, Artificial Sequence
  fluoroacetate dehalogenase mutant40(H155V,W156A)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYVASFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 86 - DNA, 900 bp, Artificial Sequence
  fluoroacetate dehalogenase mutant40(H155V,W156A)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacgttgcttctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgct

SEQ ID NO: 87 - protein, 304 aa, Artificial Sequence
  fluoroacetate dehalogenase mutant41(H155L,W156P)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYLPSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 88 - DNA, 912 bp, Artificial Sequence
  fluoroacetate dehalogenase mutant41(H155L,W156P)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacctgccgtctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 89 - protein, 304 aa, Artificial Sequence
  fluoroacetate dehalogenase mutant42(H155D,W156L)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYDLSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 90 - DNA, 912 bp, Artificial Sequence
  fluoroacetate dehalogenase mutant42(H155D,W156L)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacgacctgtctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 91 - protein, 304 aa, Artificial Sequence
  fluoroacetate dehalogenase mutant43(H155N,W156I)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYNISFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 92 - DNA, 912 bp, Artificial Sequence
  fluoroacetate dehalogenase mutant43(H155N,W156I)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacaacatctctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 93 - protein, 304 aa, Artificial Sequence
  fluoroacetate dehalogenase mutant44(H155H,W156L)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYHLSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 94 - DNA, 912 bp, Artificial Sequence
  fluoroacetate dehalogenase mutant44(H155H,W156L)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctaccacctgtctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 95 - protein, 304 aa, Artificial Sequence
  fluoroacetate dehalogenase mutant45(H155V,W156I)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYVISFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 96 - DNA, 912 bp, Artificial Sequence
  fluoroacetate dehalogenase mutant45(H155V,W156I)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacgttatctctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 97 - protein, 304 aa, Artificial Sequence
  fluoroacetate dehalogenase mutant46(H155L,W156T)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYLTSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 98 - DNA, 912 bp, Artificial Sequence
  fluoroacetate dehalogenase mutant46(H155L,W156T)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacctgacctctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 99 - protein, 304 aa, Artificial Sequence
  fluoroacetate dehalogenase mutant47(H155Q,W156I)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYQISFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 100 - DNA, 912 bp, Artificial Sequence
  fluoroacetate dehalogenase mutant47(H155Q,W156I)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctaccagatctctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 101 - protein, 304 aa, Artificial Sequence
  fluoroacetate dehalogenase mutant48(H155I,W156C)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYICSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 102 - DNA, 912 bp, Artificial Sequence
  fluoroacetate dehalogenase mutant48(H155I,W156C)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacatctgctctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 103 - protein, 304 aa, Artificial Sequence
  fluoroacetate dehalogenase mutant49(H155S,W156M)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYSMSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 104 - DNA, 912 bp, Artificial Sequence
  fluoroacetate dehalogenase mutant49(H155S,W156M)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctactctatgtctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 105 - protein, 304 aa, Artificial Sequence
  fluoroacetate dehalogenase mutant50(W156S)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYHSSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 106 - DNA, 912 bp, Artificial Sequence
  fluoroacetate dehalogenase mutant50(W156S)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctaccactcttctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 107 - protein, 304 aa, Artificial Sequence
  fluoroacetate dehalogenase mutant51(W156V)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYHVSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 108 - DNA, 912 bp, Artificial Sequence
  fluoroacetate dehalogenase mutant51(W156V)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctaccacgtttctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 109 - protein, 304 aa, Artificial Sequence
  fluoroacetate dehalogenase mutant52(H155A,W156S)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYASSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 110 - DNA, 912 bp, Artificial Sequence
  fluoroacetate dehalogenase mutant52(H155A,W156S)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacgcttcttctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 111 - protein, 304 aa, Artificial Sequence
  fluoroacetate dehalogenase mutant53(H155M,W156F)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYMFSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 112 - DNA, 912 bp, Artificial Sequence
  fluoroacetate dehalogenase mutant53(H155M,W156F)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacatgttctctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 113 - protein, 304 aa, Artificial Sequence
  fluoroacetate dehalogenase mutant54(H155E,W156T)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYETSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 114 - DNA, 912 bp, Artificial Sequence
  fluoroacetate dehalogenase mutant54(H155E,W156T)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacgaaacctctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 115 - protein, 304 aa, Artificial Sequence
  fluoroacetate dehalogenase mutant55(W156A)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYHASFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 116 - DNA, 912 bp, Artificial Sequence
  fluoroacetate dehalogenase mutant55(W156A)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctaccacgcttctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 117 - protein, 304 aa, Artificial Sequence
  fluoroacetate dehalogenase mutant56(H155E,W156M)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYEMSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 118 - DNA, 912 bp, Artificial Sequence
  fluoroacetate dehalogenase mutant56(H155E,W156M)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacgaaatgtctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 119 - protein, 304 aa, Artificial Sequence
  fluoroacetate dehalogenase mutant57(H155L,W156G)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYLGSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 120 - DNA, 912 bp, Artificial Sequence
  fluoroacetate dehalogenase mutant57(H155L,W156G)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacctgggttctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 121 - protein, 304 aa, Artificial Sequence; identical to SEQ ID NO: 77
  fluoroacetate dehalogenase mutant58(H155L,W156F)

SEQ ID NO: 122 - DNA, 912 bp, Artificial Sequence; identical to SEQ ID NO: 78
  fluoroacetate dehalogenase mutant58(H155L,W156F)

SEQ ID NO: 123 - protein, 304 aa, Artificial Sequence
  fluoroacetate dehalogenase mutant59(H155E,W156P)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYEPSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 124 - DNA, 912 bp, Artificial Sequence
  fluoroacetate dehalogenase mutant59(H155E,W156P)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacgaaccgtctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 125 - protein, 304 aa, Artificial Sequence; identical to SEQ ID NO: 87
  fluoroacetate dehalogenase mutant60(H155L,W156P)

SEQ ID NO: 126 - DNA, 912 bp, Artificial Sequence; identical to SEQ ID NO: 88
  fluoroacetate dehalogenase mutant60(H155L,W156P)

SEQ ID NO: 127 - protein, 304 aa, Artificial Sequence
  fluoroacetate dehalogenase mutant61(H155G,W156F)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYGPSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 128 - DNA, 912 bp, Artificial Sequence
  fluoroacetate dehalogenase mutant61(H155G,W156F)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacggtccgtctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

SEQ ID NO: 129 - protein, 304 aa, Artificial Sequence
  fluoroacetate dehalogenase mutant62(H155D,W156R)
MPDLADLFPGFGSEWINTSSGRIFARVGGDGPPLLLLHGFPQTHVMWHRVAPKLAERFKVIVADLPGYGWSDMPESDEQHTPYTKRAMAKQLIEAMEQLGHVHFALAGHDRGARVSYRLALDSPGRLSKLAVLDILPTYEYWQRMNRAYALKIYDRSFLAQPAPLPENLLGGDPDFYVKAKLASWTRAGDLSAFDPRAVEHYRIAFADPMRRHVMCEDYRAGAYADFEHDKIDVEAGNKIPVPMLALWGASGIAQSAATPLDVWRKWASDVQGAPIESGHFLPEEAPDQTAEALVRFFSAAPGS

SEQ ID NO: 130 - DNA, 912 bp, Artificial Sequence
  fluoroacetate dehalogenase mutant62(H155D,W156R)
atgccggacctggctgacctgttcccgggtttcggttctgaatggatcaacacctcttctggtcgtatcttcgctcgtgttggtggtgacggtccgccgctgctgctgctgcacggtttcccgcagacccacgttatgtggcaccgtgttgctccgaaactggctgaacgtttcaaagttatcgttgctgacctgccgggttacggttggtctgacatgccggaatctgacgaacagcacaccccgtacaccaaacgtgctatggctaaacagctgatcgaagctatggaacagctgggtcacgttcacttcgctctggctggtcacgaccgtggtgctcgtgtttcttaccgtctggctctggactctccgggtcgtctgtctaaactggctgttctggacatcctgccgacctacgaatactggcagcgtatgaaccgtgcttacgctctgaaaatctacgaccgttctttcctggctcagccggctccgctgccggaaaacctgctgggtggtgacccggacttctacgttaaagctaaactggcttcttggacccgtgctggtgacctgtctgctttcgacccgcgtgctgttgaacactaccgtatcgctttcgctgacccgatgcgtcgtcacgttatgtgcgaagactaccgtgctggtgcttacgctgacttcgaacacgacaaaatcgacgttgaagctggtaacaaaatcccggttccgatgctggctctgtggggtgcttctggtatcgctcagtctgctgctaccccgctggacgtttggcgtaaatgggcttctgacgttcagggtgctccgatcgaatctggtcacttcctgccggaagaagctccggaccagaccgctgaagctctggttcgtttcttctctgctgctccgggttct

(19) United States
(12) Patent Application Publication
(10) Pub. No.: US 2024/0182933 A1
(43) Pub. Date: Jun. 6, 2024

(54) FLUOROACETATE DEHALOGENASE MUTANT AND APPLICATION THEREOF

(71) Applicant: ABIOCHEM BIOTECHNOLOGY CO, LTD, Shanghai (CN)

(72) Inventors: Zhanbing CHENG, Shanghai (CN); Shaonan DING, Shanghai (CN); Yanbing XU, Shanghai (CN); Yao HUANG, Shanghai (CN)

(73) Assignee: ABIOCHEM BIOTECHNOLOGY CO, LTD, Shanghai (CN)

(21) Appl. No.: 17/787,568

(22) PCT Filed: Dec. 19, 2019
(86) PCT No.: PCT/CN2019/126785

§ 371 Date: Jun. 20, 2022

(51) Int. Cl. C12P 7/52; C12N 9/14; C12N 9/18; C12P 41/00

(52) U.S. Cl. CPC C12P 7/52; C12N 9/14; C12N 9/18; C12P 41/00; C12Y 301/01005; C12Y 308/01003

(57) ABSTRACT

Disclosed in the present invention is a fluoroacetate dehalogenase mutant, a sequence of the fluoroacetate dehalogenase mutant comprising a mutated sequence having an amino acid residue H at position 155 and/or an amino acid residue W at position 156, as shown in SEQ ID NO: 1; the fluoroacetate dehalogenase mutant has activity catalyzing bromination of a substrate, particularly a 2-bromobutyric acid substrate. Also provided in the present invention is an application of said fluoroacetate dehalogenase mutant in the preparation of (R)-2-bromobutyric acid and/or (R)-2-hydroxybutyric acid. When using the fluoroacetate dehalogenase mutant of the present invention to prepare (R)-2-bromobutyric acid, production costs are low and stereoselectivity is high, facilitating industrialized production.

### REFERENCE TO SEQUENCE LISTING

The Sequence Listing is submitted concurrently with the specification as an ASCII formatted text file via EFS-Web, with a file name of “P22412556USSEQ.TXT”, a creation date of Jun. 9, 2022, and a size of 261,208 bytes. The Sequence Listing filed via EFS-Web is part of the specification and is incorporated in its entirety by reference herein.

### TECHNICAL FIELD

The present invention belongs to the field of biotechnology, in particular, the present invention relates to a fluoroacetate dehalogenase mutant and application thereof.

### BACKGROUND

2-Oxo-1-pyrrolidine derivatives such as Levetiracetam, Brivaracetam and Seletracetam, belong to a novel antiepileptic drug developed by UCB, Belgium. EP1806339 discloses a preparation method thereof, which uses (R)-2-bromobutyric acid and corresponding 2-oxo-1-pyrrolidine compound for a substitution reaction, followed by action with triethylamine to obtain corresponding products. Among them, (R)-2-bromobutyric acid is an important raw material, which has a decisive effect on the production of antiepileptic drugs, i.e., 2-oxo-1-pyrrolidine derivatives. Thus, it is very important to find a method for the preparation of (R)-2-bromobutyric acid with low cost and high optical purity.

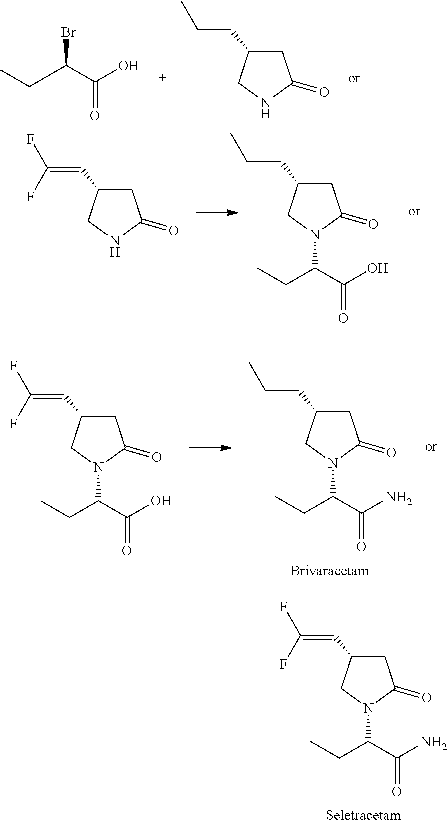

Currently, the method of preparation of (R)-2-bromobutyric acid includes a method of resolving the racemic 2-bromobutyric acid, for example, in Journal of Biological Chemistry, 1927(75), 337-365, strychnine was used as the chiral resolving agent. The method requires multiple steps of recrystallization and expensive chiral resolving agent, thereby the method is not beneficial to large-scale industrial production. Alternatively, a chiral auxiliary is used to resolve the racemic 2-bromobutyric acid, such as (S)-N-phenylpantolactone was used as the chiral auxiliary in Tetrhedron Asymmetry, 16(2005), 3739-3745. (S)-N-phenylpantolactone was esterified firstly with 2-bromobutyric acid to form an ester compound, followed by hydrolyzation to be (R)-2-bromobutyric acid. In this method, the esterification yield was only 67%, and the ee value was not desirable. Besides, the method uses a silica gel column chromatography to separate, which could not be conducted in the industrial production.

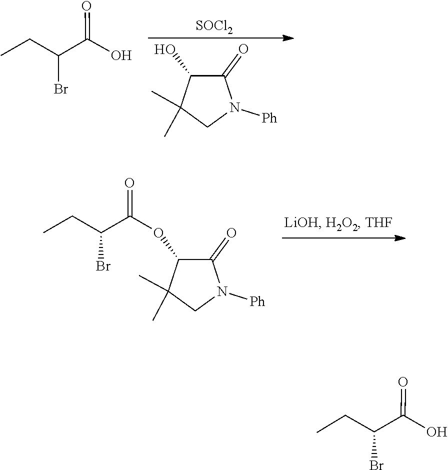

In addition, there are also chemical synthesis methods, such as in CN101048402, (R)-2-aminobutyric acid was used as raw material to react with nitrous acid and potassium bromide to obtain (R)-2-bromobutyric acid. However, this method has a very low yield, and requires the raw material to be optically pure leading to high cost.

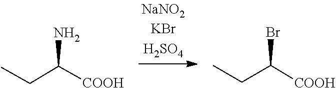

Dehalogenase is a kind of hydrolase with catalytic activity, which is capable of catalyzing the cleavage of carbon-halogen bonds in organohalogen compounds to release halogen atoms from the compounds. Dehalogenases have been reported for the resolution of halogenated compounds, e.g. J.Am.Chem.Soc., 2017, Shuguang Yuan and Manfred T. Reetz reported that fluoroacetate dehalogenase (FACD) RPA1163 could be used to obtain (R)-2-hydroxy-2-phenylacetic acid or (R)-2-hydroxy-3-phenylpropionic acid by defluorination of S-form in a racemic substrate 2-fluoro-2-phenylacetic acid or 2-fluoro-3-phenylpropionic acid, retaining unreacted (R)-2-fluoro-2-phenylacetic acid or (R)-2-fluoro-3-phenylpropionic acid to achieve the kinetic resolution. Although the enzyme is dehalogenase, the related reports only proved that the enzyme can be worked on fluorinated substrates currently, without any report that fluoroacetate dehalogenase could be used to catalyze other halogenated substrates, such as chlorinated or brominated substrates.

### Content of the Present Invention

The technical problem to be solved in the present disclosure is the defect that existing wild-type fluoroacetate dehalogenases cannot be used to catalyze brominated substrates, especially 2-bromobutyric acid substrate. Therefore, the present disclosure provides a fluoroacetate dehalogenase mutant and its application in the preparation of (R)-2-bromobutyric acid. When the fluoroacetate dehalogenase mutant of the present disclosure is used to prepare (R)-2-bromobutyric acid, the preparation has a low production cost and a high stereoselectivity, facilitating industrialized production.

The source of the wild-type fluoroacetate dehalogenase used in the present disclosure is Rhodopseudomonas palustris, the specific sequence of which is shown in the sequence listing as SEQ ID NO: 1. The wild-type fluoroacetate dehalogenase consists of 304 amino acid residues, and the applicant found that the wild-type fluoroacetate dehalogenase cannot be used to catalyze 2-bromobutyric acid by experiments. The present inventors conducted extensive screening for saturation mutations in different amino acid sites of the above wild-type enzyme for the substrate and found that some mutants with mutations at the sites H155, W156 or Y219 could be used to catalyze 2-bromobutyric acid substrate. Further, these sites were mutated combinatorially and a mutant library was constructed, from which the wild-type fluoroacetate dehalogenase described in the present disclosure was screened out.

The first technical solution of the present disclosure to solve the above technical problems is: a fluoroacetate dehalogenase mutant, wherein the fluoroacetate dehalogenase mutant comprises a sequence obtained by mutating amino acid residue H at position 155, and/or amino acid residue W at position 156 of SEQ ID NO. 1; the fluoroacetate dehalogenase mutant has activity catalyzing a brominaed substrate, particularly a 2-bromobutyric acid substrate.

Preferably, the fluoroacetate dehalogenase mutant comprises a sequence obtained by mutating amino acid residue H at position 155, and/or amino acid residue W at position 156 of SEQ ID NO. I to a natural amino acid residue.

More preferably, the fluoroacetate dehalogenase mutant further comprises a sequence obtained by mutating amino acid residue Y at position 219 of SEQ ID NO. I to a natural amino acid residue.

Further more preferably, the amino acid residue H at position 155 is mutated to A, C, D, E, F, G, I, L, M, N, P, Q ,S, T, V or W; preferably mutated to I, N, V, F, L, Q, A, C, M, P, T or W; more preferably mutated to I, N or V; and/or, the amino acid residue W at position 156 is mutated to A, C, D, F, G, I, L, M, P, R, S. T. V or Y; preferably mutated to F, M, R, S, T, G, L, A, C, D, I or Y; more preferably mutated to F. M, R, S or T; and/or, the amino acid residue Y at position 219 is mutated to a hydrophobic amino acid residue; preferably mutated to F, L or M.

Preferably, the amino acid residue H at position 155 is mutated to V, or the amino acid residue W at position 156 is mutated to M, T, C, F, S, V. A or L.

The above capitalized English single letters represent amino acids well known to those skilled in the art. According to the present disclosure, the letters herein represent corresponding amino acid residues.

Preferably, an amino acid sequence of the fluoroacetate dehalogenase mutant is shown in SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129; more preferably, a nucleotide sequence encoding the fluoroacetate dehalogenase mutant is shown in SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130.

The second technical solution of the present disclosure to solve the above technical problems is: an isolated nucleic acid, wherein the nucleic acid encodes the fluoroacetate dehalogenase mutant; preferably, the nucleotide sequence of the nucleic acid is shown in SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130.

The third technical solution of the present disclosure to solve the above technical problems is: a recombinant expression vector comprising the nucleic acid mentioned above.

The fourth technical solution of the present disclosure to solve the above technical problems is: a transformant comprising the nucleic acid or the recombinant expression vector mentioned above.

The fifth technical solution of the present disclosure to solve the above technical problems is: a dehalogenase combination catalyzing a halogenated substrate, wherein the dehalogenase combination catalyzing a halogenated substrate comprises two or more of fluoroacetate dehalogenases or mutants thereof, wherein the fluoroacetate dehalogenase mutants are the fluoroacetate dehalogenase mutant of the present disclosure. Preferably, the halogenated substrate is a brominated substrate; more preferably a 2-bromobutyric acid, methyl 2-bromobutyrate or ethyl 2-bromobutyrate substrate.

The sixth technical solution of the present disclosure to solve the above technical problems is: a catalyst combination, wherein the catalyst combination comprises (a) the fluoroacetate dehalogenase mutant or the dehalogenase combination catalyzing a halogenated substrate of the present disclosure, and (b) a lipase, wherein the lipase is preferably lipase B from Candida antartica.

The seventh technical solution of the present disclosure to solve the above technical problems is: a reaction system for a catalyst, wherein the reaction system comprises a substrate and a catalyst, wherein the substrate is 2-bromobutyric acid, methyl 2-bromobutyrate or ethyl 2-bromobutyrate, wherein,
(a) when the substrate is 2-bromobutyric acid, the catalyst comprises the fluoroacetate dehalogenase mutant or the dehalogenase combination catalyzing a halogenated substrate of the present disclosure; wherein,
the 2-bromobutyric acid has a concentration of 0.02 g/ml-0.2 g/ml, preferably 0.1-0.15 g/ml, more preferably 0.1 g/ml;
the fluoroacetate dehalogenase or mutant thereof has a concentration of 2-20 U/ml, preferably 10 U/ml;
(b) when the substrate is methyl 2-bromobutyrate or ethyl 2-bromobutyrate, the catalyst comprises the catalyst combination of the present disclosure; wherein, the methyl 2-bromobutyrate or ethyl 2-bromobutyrate has concentration of 0.02 g/ml-0.2 g/ml, preferably 0.1-0.15 g/ml, more preferably 0.15 g/ml;
the fluoroacetate dehalogenase or mutant thereof has a concentration of 2-20 U/ml, preferably 10 U/ml;
the lipase has a concentration of 1-20 g/L, preferably 10 g/L;
when the reaction system comprises a lipase, the reaction system further comprises methanol, wherein a volume ratio of the methanol to the reaction system is 1-10%, preferably 10%.

In a preferred embodiment of the present disclosure, the reaction system further comprises a reaction solvent, wherein the reaction solvent is preferably water.

In another preferred embodiment of the present disclosure, pH is 6-8, preferably 7. When the pH is too high, the feedstock will decompose spontaneously, which will affect the yield.

According to the present disclosure, the substrate can be one or more of racemate, R-form or S-form enriched enantiomer, wherein the R-form-enriched means that the R-form coexists with the S-form and the molar ratio of R-form to S-form is more than 1:1, and the S-form-enriched means that the R-form coexists with the S-form and the molar ratio of S-form to R-form is more than 1:1.

The eighth technical solution of the present disclosure to solve the above technical problems is: a use of the fluoroacetate dehalogenase mutant, the dehalogenase combination catalyzing a halogenated substrate, the catalyst combination, the reaction system in preparation of (R)-2-bromobutyric acid and/or (R)-2-hydroxybutyric acid.

The ninth technical solution of the present disclosure to solve the above technical problems is: a preparation method of (R)-2-bromobutyric acid, wherein the preparation method comprises following steps: a catalytic reaction is performed in the presence of the reaction system mentioned above to obtain (R)-2-bromobutyric acid. Herein the catalytic reaction comprises a dehalogenation reaction, or a dehalogenation reaction and an ester hydrolysis reaction.

Herein, preferably, the catalytic reaction has a reaction system with a temperature of 20-30° C., preferably 30° C.;
and/or, the catalytic reaction has a reaction time of 5-10 hours, preferably 8 hours.

When the substrate is 2-bromobutyric acid, the reaction formula is as follows:

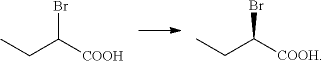

The tenth technical solution of the present disclosure to solve the above technical problems is: a preparation method of (R)-2-hydroxybutyric acid, wherein the preparation method comprises following steps: a catalytic reaction is performed in the presence of the reaction system mentioned above to obtain (R)-2-hydroxybutyric acid. Herein the catalytic reaction comprises a dehalogenation reaction, or a dehalogenation reaction and an ester hydrolysis reaction.

When the substrate is 2-bromobutyric acid, the dehalogenase reaction is highly selective for (S)-2-bromobutyric acid in 2-bromobutyric acid (i.e., the dehalogenase preferentially reacts with (S)-2-bromobutyric acid to generate (R)-2-hydroxybutyric acid, and after the (S)-2-bromobutyric acid reacts completely, the (R)-2-bromobutyric acid participates in the reaction), resulting in dehalogenation and configuration inversion to obtain (R)-2-hydroxybutyric acid, while the unreacted or incomplete reacted (R)-2-bromobutyric acid in 2-bromobutyric acid is retained. The physicochemical properties of (R)-2-bromobutyric acid and (R)-2-hydroxybutyric acid in the product are quite different, so they are easily to be separated.

Preferably, the catalytic reaction has a reaction system with a temperature of 20-30° C., preferably 30° C.; when the temperature is too high, the feedstock will decompose spontaneously, which will affect the yield;
and/or, the catalytic reaction has a reaction time of 5-10 hours, preferably 8 hours.

When the substrate is 2-bromobutyric acid, the reaction formula is as follows:

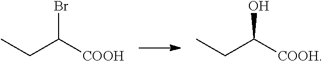

Unless otherwise specified, the concentration of the above compounds shall be the concentration of the above compounds in the whole reaction system before the reaction.

On the basis of common knowledge in the art, each of the above preferred conditions can be combined in any way to obtain each preferred example of the present invention.

The reagents and raw materials used in the present invention are commercially available.

The positive progressive effect of the present invention is that:

The fluoroacetate dehalogenase mutant of the present disclosure can be used to catalyze brominated substrates, in particular 2-bromobutyric acid. In a preferred example of the present disclosure, the fluoroacetate dehalogenase mutant is capable of catalyzing a substrate with a concentration of up to 0.1 g/ml. When the fluoroacetate dehalogenase mutant of the present disclosure is used to prepare (R)-2-bromobutyric acid, the preparation has a low cost and a high stereoselectivity, facilitating industrialized production.

### BRIEF DESCRIPTION OF THE DRAWINGS

FIG. 1 is the results of chiral HPLC analysis of the reaction feedstock 2-bromobutyric acid in Example 3.

FIG. 2 is the results of chiral HPLC analysis of (R)-2-bromobutyric acid after the reaction involving fluoroacetate dehydrogenase mutant 3 in Example 3.

FIG. 3 is the results of chiral HPLC analysis of (R)-2-bromobutyric acid standard.

FIG. 4 is the HPLC analysis spectrum of the reaction feedstock 2-bromobutyric acid in Example 3.

FIG. 5 is the HPLC analysis spectrum of the racemic 2-hydroxybutyric acid standard.

FIG. 6 is the HPLC analysis spectrum of the reaction solution after the reaction involving fluoroacetate dehydrogenase mutant 3 in Example 3.

### DETAILED DESCRIPTION OF THE PREFERRED EMBODIMENT

The following examples further illustrate the present disclosure, but the present disclosure is not limited thereto. Experimental methods for which specific conditions are not indicated in the following examples are selected according to conventional methods and conditions, or according to the trade description.

The experimental methods in the present invention are conventional unless otherwise specified, and the gene cloning operations can be specified in Molecular Cloning: A Laboratory Manual, edited by J Sambrook et al.

The 2-bromobutyric acid in the present disclosure can be one or more of 2-bromobutyric acid racemate, R-form-enriched 2-bromobutyric acid and S-form-enriched 2-bromobutyric acid unless otherwise specified, wherein the R-form-enriched means that the R-form coexists with the S-form and the molar ratio of R-form to S-form is more than 1:1, and the S-form-enriched means that the R-form coexists with the S-form and the molar ratio of S-form to R-form is more than 1:1.

The amino acid abbreviation symbols in the present disclosure are conventional in the art unless otherwise specified, and the amino acids corresponding to the specific abbreviation symbols are shown in Table 1.

TABLE 1

| Amino | Three | Single | | Three | Single |
|---|---|---|---|---|---|
| acid | letters | letter | Amino | letters | letter |
| name | symbol | symbol | acid name | symbol | symbol |
| Alanine | Ala | A | Leucine | Leu | L |
| Arginine | Arg | R | Lysine | Lys | K |
| Asparagine | Asn | N | Methionine | Met | M |
| Aspartic acid | Asp | D | Phenylalanine | Phe | F |
| Cysteine | Cys | C | Proline | Pro | P |
| Glutanine | Gln | Q | Serine | Ser | S |
| Glutamic acid | Glu | E | Threonine | Thr | T |
| Glycine | Gly | G | Tryptophan | Trp | W |
| Histidine | His | H | Tyrosine | Tyr | Y |
| Isoleucine | Ile | I | Valine | Val | V |

The codons corresponding to the amino acids are also conventional in the art, and the corresponding relationships of specific amino acids with the codons are shown in Table 2.

TABLE 2

| The first | The second nucleotide | | | | The third |
|---|---|---|---|---|---|
| nucleotide | T | C | A | G | nucleotide |
| T | Phenylalanine (F) | Serine (S) | Tyrosine (Y) | Cysteine (C) | T |
| | Phenylalanine (F) | Serine (S) | Tyrosine (Y) | Cysteine (C) | C |
| | Leucine (L) | Serine (S) | Stop codon | Stop codon | A |
| | Leucine (L) | Serine (S) | Stop codon | Tryptophan (W) | G |
| C | Leucine (L) | Proline (P) | Histidine (H) | Arginine (R) | T |
| | Leucine (L) | Proline (P) | Histidine (H) | Arginine (R) | C |
| | Leucine (L) | Proline (P) | Glutamine (Q) | Arginine (R) | A |
| | Leucine (L) | Proline (P) | Glutamine (Q) | Arginine (R) | G |
| A | Isoleucine (I) | Threonine (T) | Asparagine (N) | Serine (S) | T |
| | Isoleucine (I) | Threonine (T) | Asparagine (N) | Serine (S) | C |
| | Isoleucine (I) | Threonine (T) | Lysine (K) | Arginine (R) | A |
| | Methionine (M) | Threonine (T) | Lysine (K) | Arginine (R) | G |
| G | Valine (V) | Alanine (A) | Aspartic acid (D) | Glycine (G) | T |
| | Valine (V) | Alanine (A) | Aspartic acid (D) | Glycine (G) | C |
| | Valine (V) | Alanine ( A) | Glutamic acid (E) | Glycine (G) | A |
| | Valine (V) | Alanine (A) | Glutamic acid (E) | Glycine (G) | G |

pET28a and bugbuster protein extraction reagent were purchased from Novagen; enzyme DpnI was purchased from Invitrogen Shanghai Trading Co., Ltd; E. coli BL21(DE3) competent cell was purchased from Beijing Dingguo Changsheng Biotech Co., Ltd; 2-bromobutyric acid was purchased from Yancheng City Shengda Chemical Co., Ltd.

The method of chiral HPLC (High Performance Liquid Chromatography) analysis for the substrate 2-bromobutyric acid and the product 2-bromobutyric acid is as follows:

Chromatographic condition: Daicel Chiralpak IG, 4.6 mm*250 mm, 5 μm; mobile phase: n-hexane/isopropanol/TFA=99:1:0.1; wavelength of detection: 230 nm; flow rate: 0.7 mL/min; temperature of column: 25° C.

The method of HPLC analysis for the substrate 2-bromobutyric acid and the product is as follows:

Chromatographic condition: Inertsil ODS-3, 4.6 mm*150 mm, 5 μm; mobile phase A: 10 mmol sodium dihydrogen phosphate aqueous solution (pH=3.0), mobile phase B: acetonitrile; gradient elution; wavelength of detection: 205nm; flow rate: 0.7 mL/min; temperature of column: 35° C.

Conversion rate=(Reagent−Remaining Reagent)/Reagent×100% (Reagent: 2-bromobutyric acid).

### Example 1 Construction of the Fluoroacetate Dehalogenase Mutant Library

The primer sequences designed for the construction of the mutant library with mutations at positions 155, 156, and 219 in the sequence of the fluoroacetate dehalogenase FACD-RPA1163 (the source is Rhodopseudomonas palustris) (i.e., SEQ ID NO: 1 in the sequence listing) are shown in Table 3:

TABLE 3

| | Mutation positions | | SEQ ID |
|---|---|---|---|
| No. | and primer names | primer sequence | NO: |
| 1 | H155W156-Forward | GCACTGAAAATCTATNNKNNKAGCTTTCTGGCACAG | 3 |
| | primer | C | |
| 2 | H155W156-Reverse | GCTGTGCCAGAAAGCTMNNMNNATAGATTTTCAGT | 4 |
| | primer | GC | |
| 3 | Y219-Forward primer | GTGATGTGTGAAGATNNKCGTGCGGGTGCGTATG | 5 |
| 4 | Y219-Reverse primer | CATACGCACCCGCACGMNNATCTTCACACATCAC | 6 |

Wherein N represents any one of nucleotides A, G, C and T, M represents A or C, and K represents G or T; which are selected according to the coding nucleotide of the amino acid to be mutated at the position. For example, NNK in A166-Forward primer can represent AAG (Lysine), AAT (Aspartic acid), AGG (Arginine) or AGT (Serine), etc. The nucleotides corresponding to the specific amino acids can refer to Table 2.

The plasmid template pET28a-FACD-RPA1163 was constructed according to the method disclosed in J. Am. Chem. Soc., 2017,139(32), 11241-11247, and the target bands were amplified by PCR with pET28a-FACD-RPA1163 as the template. The amplification reaction system was:

50 μL PCR amplification system:

| Reagents | Dosage (μL) |
|---|---|
| 2 × PCR buffer ( with high fidelity enzyme) | 25 |
| Primer F | 1 |
| Primer R | 1 |
| Template | 1 |
| Deionized water | 22 |

The amplification procedure was as follows:

| | | | |
|---|---|---|---|
| 95° C. | 5 min | | |
| 95° C. | 30 s | | |
| 50° C. | 30 s | {close oversize brace} | 30 cycles |
| 72° C. | 5 min | | |
| 72° C. | 10 min | | |
| 12° C. | Hold | | |

The PCR products were digested by DpnI at 37° C. for 2 hr, followed by being transformed into E. coli BL21 (DE3) competent cells after the reaction. The cells were coated on LB medium containing 100 μg/mL Ampicillin, and incubated overnight at 37° C. to obtain transformants containing the mutant library.

### Example 2 Screening the Mutant Library in High-Throughput

The screening was performed according to the following experimental steps:

The transformants obtained in Example 1 were inoculated in the 96-well plate and induced by IPTG overnight at 30° C. After that, the bacteria were collected, lysed by adding bugbuster protein extraction reagent and centrifuged to obtain the total enzyme solution.

250 mg of substrate racemic 2-bromobutyric acid was taken to be dissolved in 45 mL of 1 mM Tris solution, and the pH was adjusted to 8.0-8.5 with dilute NaOH on ice, and the volume was set to 50 ml to prepare the substrate solution with the substrate 2-bromobutyric acid in a concentration of 30 mM. 100 μl of the substrate solution (final concentration of 25 mM) and 20 μL of the above total enzyme solution were added in each reaction. After the solution was reacted for a period of time, 20 μl of the reaction solution was added into the 96-well ELISA plate, 30 μl of saturated Hg(SCN)₂ ethanol solution and 150 μL of 60 g/L FeNH₄(SO₄)₂·12H₂O (containing 1% nitric acid, w/v) aqueous solution were added sequentially. After 10 min at room temperature, the absorbance was detected at 500 nm by a Microplate Reader.

The wild type enzyme was used as the reference system (the activity of the wild type enzyme could not be detected, which shows that the wild type fluoroacetate dehalogenase FAcD-RPA1163 has no catalytic effect on 2-bromobutyric acid), and positive clones were selected, sequenced and the enzyme activity of which was detected. The sequences of the positive clones were determined by sequencing at Sangon Biotech (Shanghai) Co., Ltd, 698 Xiangmin Road, Songjiang District, Shanghai.

The positive clones were selected and cultured as follows:

10% of the above positive clones were inoculated in 150 mL TB medium and incubated at 37° C. until the OD₆₀₀ value reached about 0.8. Then IPTG was added until the final concentration thereof is 0.1 mM and cooled to 30° C. for induction overnight. After incubation, the culture medium was centrifuged at 4000 rpm for 30 min, the supernatant was discarded, and the bacteria were collected and stored in an ultra-low temperature refrigerator at −80° C. for later use.

The bacteria collected at the end of the culture were washed twice with 20 mM pH 8.0 phosphate buffer, after which the bacteria were resuspended in phosphate buffer with pH 8.0, homogenized and crushed under low temperature and high pressure, and the crushed solution was centrifuged to remove the precipitate, and the obtained supernatant was the mutant crude enzyme solution containing the recombinant dehalogenase.

The above methods were used for the preparation of the crude enzyme solution of the dehalogenase mutant in the following examples.

The method of the enzyme activity assay is: 2.5 g of bacteria were resuspended with 40 mL of 20 mM pH 8.0 phosphate buffer, homogenized and crushed under low temperature and high pressure, the precipitate was discarded by centrifugation, and the supernatant was left to obtain the crude enzyme solution. 900 μL of the substrate solution (30 mM) was added at 30° C., and 100 μL of the crude enzyme solution to be tested was added to obtain 1 mL of the reaction solution. 150 μl of saturated Hg(SCN)₂ ethanol solution and 750 μL of 60 g/L FeNH₄(SO₄)₂·12H₂O (containing 1% nitric acid, w/v) aqueous solution were added into 100 μL of the reaction solution sequentially. After 10 min at room temperature, the absorbance value was detected at 500 nm with an UV spectrophotometer. The enzyme activity was calculated based on the production rate of bromine ion. The results are shown in Table 4.

The unit enzyme activity is defined as: the amount of enzyme required to produce 1 μmol of bromine ion per minute under specific reaction conditions (30° C.)

TABLE 4

| fluoroacetate | | | Amino | Nucleotide |
|---|---|---|---|---|
| dehalogenase | | Enzyme | acid SEQ | SEQ |
| mutant No. | Mutation position | activity | ID NO: | ID NO: |
| 1 | H155I, W156S, Y219F | ***** | 7 | 8 |
| 2 | H155V, W156M, Y219L | ***** | 9 | 10 |
| 3 | H155V, W156R, Y219M | ***** | 11 | 12 |
| 4 | H155I, W156S | ***** | 13 | 14 |
| 5 | H155N, W156F | ***** | 15 | 16 |
| 6 | H155V, W156T | ***** | 17 | 18 |
| 7 | W156M | ***** | 19 | 20 |
| 8 | H155V, W156M | ***** | 21 | 22 |
| 9 | H155V, W156R | ***** | 23 | 24 |
| 10 | H155Q, W156F | **** | 25 | 26 |
| 11 | H155L, W156S | **** | 27 | 28 |
| 12 | H155F, W156G | **** | 29 | 30 |
| 13 | H155F, W156L | **** | 31 | 32 |
| 14 | H155Q, W156L | *** | 33 | 34 |
| 15 | H155V, W156D | *** | 35 | 36 |
| 16 | H155M, W156A | *** | 37 | 38 |
| 17 | H155L, W156I | *** | 39 | 40 |
| 18 | H155F, W156M | *** | 41 | 42 |
| 19 | H155F, W156R | *** | 43 | 44 |
| 20 | H155A, W156F | *** | 45 | 46 |
| 21 | H155P, W156Y | *** | 47 | 48 |
| 22 | W156T | *** | 49 | 50 |
| 23 | H155V | *** | 51 | 52 |
| 24 | W156C | *** | 53 | 54 |
| 25 | H155T, W156F | *** | 55 | 56 |
| 26 | H155W, W156I | *** | 57 | 58 |
| 27 | H155L, W156L | *** | 59 | 60 |
| 28 | H155P, W156T | *** | 61 | 62 |
| 29 | H155C, W156M | *** | 63 | 64 |
| 30 | H155I, W156A | ** | 65 | 66 |
| 31 | H155L, W156A | ** | 67 | 68 |
| 32 | W156F | ** | 69 | 70 |
| 33 | H155T, W156L | ** | 71 | 72 |
| 34 | H155V, W156Y | ** | 73 | 74 |
| 35 | H155I, W156P | ** | 75 | 76 |
| 36 | H155L, W156F | ** | 77 | 78 |
| 37 | H155V, W156G | ** | 79 | 80 |
| 38 | H155F, W156T | ** | 81 | 82 |
| 39 | H155A, W156M | ** | 83 | 84 |
| 40 | H155V, W156A | ** | 85 | 86 |
| 41 | H155L, W156P | ** | 87 | 88 |
| 42 | H155D, W156L | ** | 89 | 90 |
| 43 | H155N, W156I | ** | 91 | 92 |
| 44 | W156L | ** | 93 | 94 |
| 45 | H155V, W156I | ** | 95 | 96 |
| 46 | H155L, W156T | ** | 97 | 98 |
| 47 | H155Q, W156I | ** | 99 | 100 |
| 48 | H155I, W156C | ** | 101 | 102 |
| 49 | H155S, W156M | * | 103 | 104 |
| 50 | W156S | * | 105 | 106 |
| 51 | W156V | * | 107 | 108 |
| 52 | H155A, W156S | * | 109 | 110 |
| 53 | H155M, W156F | * | 111 | 112 |
| 54 | H155E, W156T | * | 113 | 114 |
| 55 | W156A | * | 115 | 116 |
| 56 | H155E, W156M | * | 117 | 118 |
| 57 | H155L, W156G | * | 119 | 120 |
| 58 | H155L, W156F | * | 121 | 122 |
| 59 | H155E, W156P | * | 123 | 124 |
| 60 | H155L, W156P | * | 125 | 126 |
| 61 | H155G, W156F | * | 127 | 128 |
| 62 | H155D, W156R | * | 129 | 130 |
| 63 | WT | — | 1 | 2 |

Wherein no enzyme activity could be detected in wild-type WT, *represents enzyme activity is below 0.1 U/ml; **represents enzyme activity is between 0.1-1 U/ml: *** represents enzyme activity is between 1-5 U/ml; ****represents enzyme activity is between 5-10 U/ml; *****represents enzyme activity is between 10 U/mL-20 U/mL.

### Example 3 Preparation of (R)-2-Bromobutyric Acid Catalyzed by the Fluoroacetate Dehalogenase Mutant

50 mL of tap water and 10 g of the substrate 2-bromobutyric acid were added into the reaction flask, and stirred to dissolve. The pH was adjusted to 7.0 with 30% NaOH solution. 20 mL of the mutant crude enzyme solution prepared according to the method in Example 2 (i.e., 20 mL of 100 mM pH 7.0 disodium hydrogen phosphate-sodium dihydrogen phosphate buffer was added into 4 g of the bacteria described in Example 2, stirred well, then homogenized and crushed under high pressure to obtain the mutant crude enzyme solution. Take fluoroacetate dehalogenase mutant 3 as an example, the concentration of which was 10 U/ml) was added, and the volume was set to 100 mL by tap water. The pH was controlled at about 7.0 with 2 mol/L sodium carbonate solution during the reaction. After reacted in water bath at 30° C. for 8 hours, the conversion rate of the substrate 2-bromobutyric acid was detected by sampling. The reaction solution has a pH of 1-2 adjusted with 40% aqueous sulfuric acid, filtered with diatomaceous earth, and the filtrate was extracted three times with an equal volume of toluene. The toluene was combined, and concentrated at 50° C. to obtain (R)-2-bromobutyric acid. The ee value of (R)-2-bromobutyric acid was detected by HPLC. The results of the conversion rate and the ee value are shown in Table 5. The aqueous phase has a pH of 0-1 adjusted with 40% aqueous sulfuric acid, then extracted three times with an equal volume of methyl tert-butyl ether. The methyl tert-butyl ether was combined, and concentrated at 50° C. to obtain 2-hydroxybutyric acid. The optical rotation of 2-hydroxybutyric acid was [a]25D=+4.2° (C=3.0, H₂O). The product could be known as (R)-2-hydroxybutyric acid by referring to the content of Advanced Synthesis and Catalysis, 2016, vol. 358, #18, p. 2923-2928.

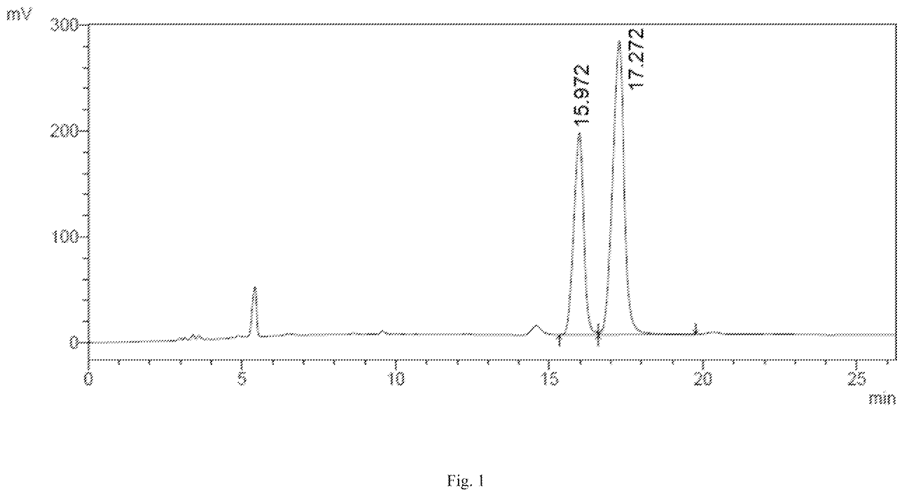

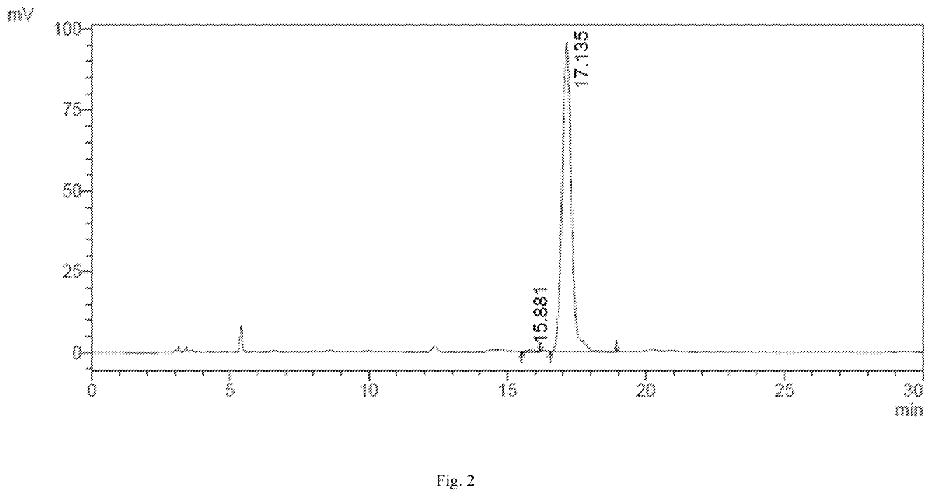

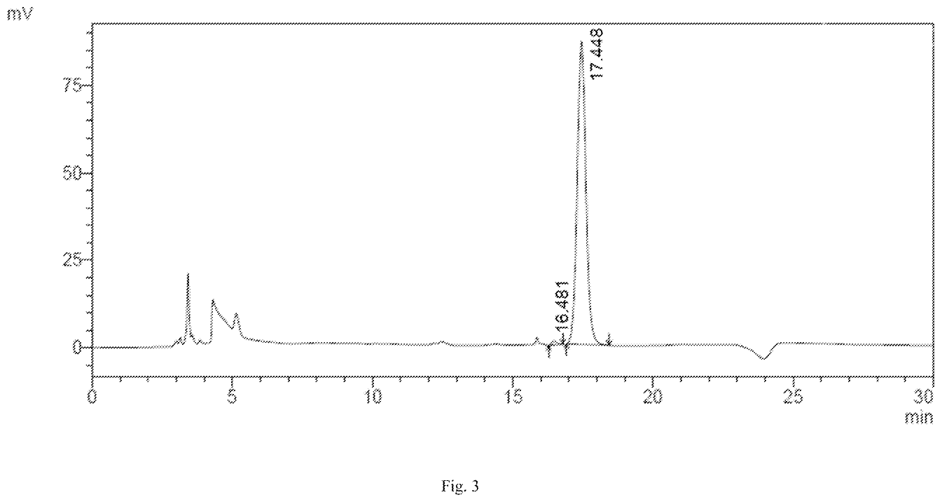

The results of the chiral HPLC analysis of 2-bromobutyric acid used as the reaction feedstock are shown in FIG. 1, wherein (R)-2-bromobutyric acid has a retention time of 17.272 min, and (S)-2-bromobutyric acid has a retention time of 15.972 min. The results of the chiral HPLC analysis of (R)-2-bromobutyric acid and (S)-2-bromobutyric acid in the post-reaction products are shown in FIG. 2 (the figures are all illustrated with fluoroacetate dehalogenase mutant 3 as an example), wherein (R)-2-bromobutyric acid has a retention time of 17.135 min and (S)-2-bromobutyric acid was almost undetectable. FIG. 3 is the chiral HPLC spectrum of (R)-2-bromobutyric acid standard, which was prepared in our laboratory according to CN101048402, and its HNMR identification data is ¹HNMR(400 MHZ, CDCl₃) δ 4.209-4.173; (t, 1H), δ2.130-2.004; (m, 2H), δ1.081-1.044; (t, 3H). The component of the product prepared in this example has basically the same as the peak time of (R)-2-bromobutyric acid standard, indicating that almost all of the (S)-2-bromobutyric acid was reacted in this example, i.e., all of the (S)-2-bromobutyric acid was converted into (R)-2-hydroxybutyric acid.

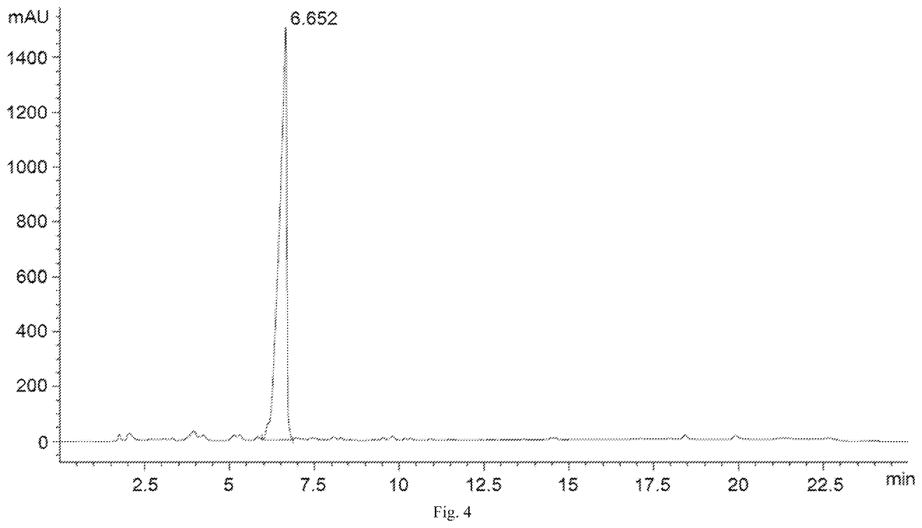

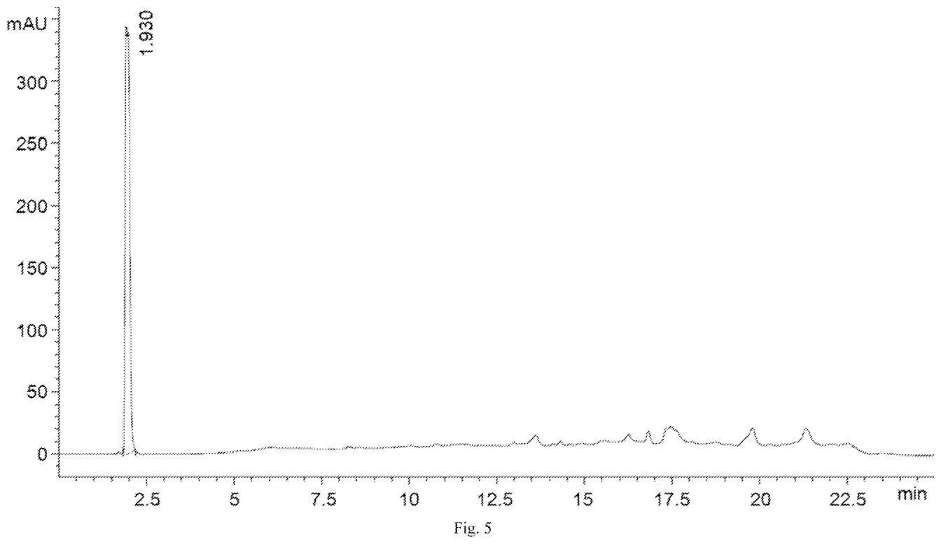

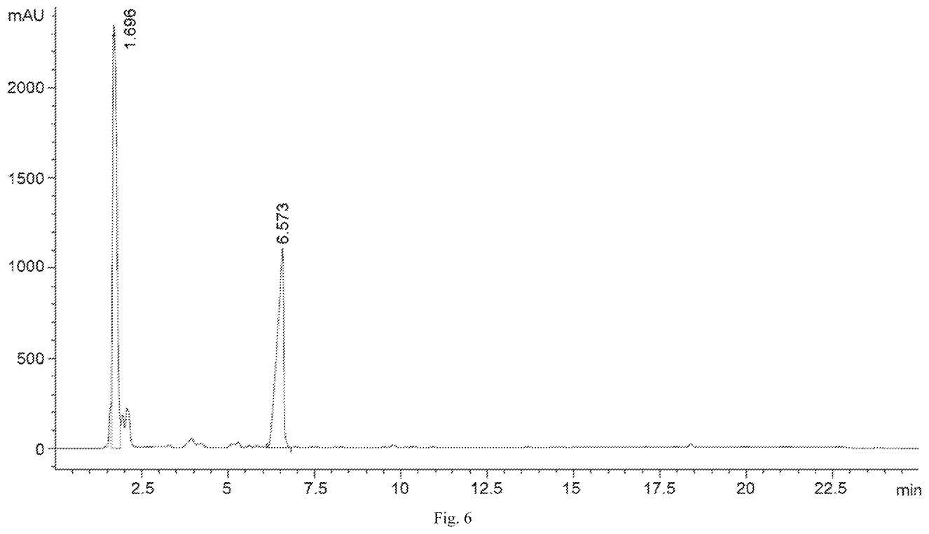

The results of HPLC chromatogram analysis of 2-bromobutyric acid used in the reaction feedstock are shown in FIG. 4, wherein 6.652 min was the peak position of 2-bromobutyric acid. The HPLC chromatogram analysis results of the racemic 2-hydroxybutyric acid standard (purchased from Shanghai Titan Chemical Co., Ltd) are shown in FIG. 5, wherein the retention time of 2-hydroxybutyric acid standard was 1.930 min. The results of HPLC chromatogram analysis of the reaction solution after the reaction are shown in FIG. 6. It can be seen that the peak times of 2-bromobutyric acid and (R)-2-hydroxybutyric acid in the products after the reaction were basically the same as those of the respective standards.

TABLE 5

| Fluoroacetate | | ee value ((R)-2- |
|---|---|---|
| dehalogenase | Conversion | bromobutyric |
| mutant No. | rate | acid) |
| 1 | 52% | 99% |
| 2 | 54% | 60% |
| 3 | 60% | 100% |
| 4 | 56% | 100% |
| 5 | 58% | 100% |
| 6 | 56% | 99.12% |
| 7 | 56% | 98.32% |
| 8 | 56% | 99.72% |
| 9 | 55% | 97.1% |
| 10 | 49% | 93.67% |
| 11 | 48% | 93.56 |
| 12 | 48% | 90.84 |
| 13 | 48% | 90.63 |
| 14 | 47% | 89.92 |
| 15 | 46% | 88.87 |
| 16 | 46% | 88.26 |
| 17 | 46% | 88.22 |
| 18 | 46% | 86.17 |
| 19 | 46% | 85.18 |
| 20 | 46% | 84.75 |
| 21 | 46% | 84 |
| 22 | 46% | 83.66 |
| 23 | 45% | 81.91 |
| 24 | 45% | 81.76 |
| 25 | 45% | 81.64 |
| 26 | 45% | 81.35 |
| 27 | 45% | 80.93 |
| 28 | 45% | 80.81 |
| 29 | 45% | 80.53 |
| 30 | 44% | 79.58 |
| 31 | 44% | 78.61 |
| 32 | 44% | 78.5 |
| 33 | 44% | 78.44 |
| 34 | 44% | 77.41 |
| 35 | 43% | 76.89 |
| 36 | 43% | 76.82 |
| 37 | 43% | 76.77 |
| 38 | 43% | 76.73 |
| 39 | 43% | 76.69 |
| 40 | 43% | 76.36 |
| 41 | 43% | 75.39 |
| 42 | 43% | 75.13 |
| 43 | 43% | 74.98 |
| 44 | 42% | 73.86 |
| 45 | 42% | 73.79 |
| 46 | 42% | 73.38 |
| 47 | 42% | 72.32 |
| 48 | 42% | 71.27 |
| 49 | 41% | 69.72 |
| 50 | 41% | 68.78 |
| 51 | 40% | 67.39 |
| 52 | 40% | 67.19 |
| 53 | 40% | 66.94 |
| 54 | 40% | 66.26 |
| 55 | 40% | 66.23 |
| 56 | 40% | 65.44 |
| 57 | 39% | 65.12 |
| 58 | 39% | 64.68 |
| 59 | 39% | 63.54 |
| 60 | 39% | 63.53 |
| 61 | 38% | 62.25 |
| 62 | 37% | 59.22 |

### Example 4 Preparation of (R)-2-bromobutyric Acid Catalyzed by the Fluoroacetate Dehalogenase Mutant and Lipase by One-Pot Process

40 mL of tap water, 10mL of methanol and 15 g of the substrate methyl 2-bromobutyrate were added into the reaction flask, stirred to dissolve, and the pH of which was controlled at 7.0. 20 mL of the mutant crude enzyme solution prepared according to the method in Example 2 (i.e., 20 mL of 100 mM pH 7.0 disodium hydrogen phosphate-sodium dihydrogen phosphate buffer was added into 4 g of the bacteria described in Example 2, stirred well, then homogenized and crushed under high pressure to obtain the mutant crude enzyme solution, take fluoroacetate dehalogenase mutant 3 as an example, the concentration of which was 10 U/ml) next 1 g of lipase Novozyme® 435 (the lipase B from Candida antartica (CALB), which was immobilized in macroporous polymethylmethacrylate resin (Lewatit), and its concentration in this reaction could be 1-20 g/L) were added, and finally the volume of the reaction system was set as 100 mL by tap water. The pH was controlled at about 7.0 with 2 mol/L sodium carbonate solution during the reaction. After reacted in water bath at 30° C. for 8 hours, the conversion rate of the reaction was detected by sampling.

The reaction solution has a pH of 1-2 adjusted with 40% aqueous sulfuric acid, filtered with diatomaceous earth, and the filtrate was extracted three times with an equal volume of toluene. The toluene was combined, and concentrated at 50° C. to obtain 3.5 g of (R)-2-bromobutyric acid. The ee value of (R)-2-bromobutyric acid was detected by HPLC and the ee value was 98%. The aqueous phase has a pH of 0-1 adjusted with 40% aqueous sulfuric acid, then extracted three times with an equal volume of methyl tert-butyl ether. The methyl tert-butyl ether was combined and concentrated at 50° C. to obtain 2-hydroxybutyric acid. The optical rotation of 2-hydroxybutyric acid was determined as [a]25D=+4.1° (C=3.0, H₂O). The product could be known as (R)-2-hydroxybutyric acid by referring to the content of Advanced Synthesis and Catalysis, 2016, vol. 358, #18, p. 2923-2928.

Although the above describes specific embodiments of the present disclosure, it should be understood by a person skilled in the art that these are merely illustrative examples and that a variety of changes or modifications can be made to these embodiments without departing from the principles and substance of the present invention. Therefore, the scope of protection of the present disclosure is limited by the appended claims.

1. A fluoroacetate dehalogenase mutant, wherein the fluoroacetate dehalogenase mutant comprises a sequence obtained by mutating amino acid residue H at position 155, and/or amino acid residue W at position 156 of SEQ ID NO. 1; the fluoroacetate dehalogenase mutant has activity catalyzing a brominated substrate, particularly a 2-bromobutyric acid substrate.

2. The fluoroacetate dehalogenase mutant of claim 1, wherein the fluoroacetate dehalogenase mutant comprises a sequence obtained by mutating amino acid residue H at position 155, and/or amino acid residue W at position 156 of SEQ ID NO. 1 to a natural amino acid residue;
preferably, the fluoroacetate dehalogenase mutant further comprises a sequence obtained by mutating amino acid residue Y at position 219 of SEQ ID NO. 1 to a natural amino acid residue.

3. The fluoroacetate dehalogenase mutant of claim 2, wherein the amino acid residue H at position 155 is mutated to A, C, D, E, F, G, I, L, M, N, P, Q, S, T, V or W; and/or, the amino acid residue W at position 156 is mutated to A, C, D, F, G, I, L, M, P, R, S, T, V or Y; and/or, the amino acid residue Y at position 219 is mutated to a hydrophobic amino acid residue;
preferably, the amino acid residue H at position 155 is mutated to I, N, V, F, L, Q, A, C, M, P, T or W; and/or, the amino acid residue W at position 156 is mutated to F, M, R, S, T, G, L, A, C, D, I or Y; and/or, the amino acid residue Y at position 219 is mutated to F, L or M;
more preferably, the amino acid residue H at position 155 is mutated to I, N or V; and/or, the amino acid residue W at position 156 is mutated to F, M, R, S or T; and/or, the amino acid residue Y at position 219 is mutated to F, L or M;
more preferably, the amino acid residue H at position 155 is mutated to V, or the amino acid residue W at position 156 is mutated to M, T, C, F, S, V, A or L.

4. (canceled)

5. The fluoroacetate dehalogenase mutant of claim 1, wherein an amino acid sequence of the fluoroacetate dehalogenase mutant is shown in SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO:
117, SEQ ID NO: 119, SEQ ID NO: 121, SEQ ID NO: 123, SEQ ID NO: 125, SEQ ID NO: 127, SEQ ID NO: 129; preferably, a nucleotide sequence of the fluoroacetate dehalogenase mutant is shown in SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130.

6. An isolated nucleic acid, wherein the nucleic acid encodes the fluoroacetate dehalogenase mutant of claim 1.

7. A recombinant expression vector comprising the nucleic acid of claim 6.

8. A transformant comprising the nucleic acid of claim 6.

9. A dehalogenase combination catalyzing a halogenated substrate, wherein the dehalogenase combination catalyzing a halogenated substrate comprises two or more of fluoroacetate dehalogenases or mutants thereof, wherein the fluoroacetate dehalogenase mutants are the fluoroacetate dehalogenase mutant of claim 1; preferably, the halogenated substrate is a brominated substrate; more preferably a 2-bromobutyric acid, methyl 2-bromobutyrate or ethyl 2-bromobutyrate substrate.

10. A catalyst combination, wherein the catalyst combination comprises (a) the fluoroacetate dehalogenase mutant of claim 1, and (b) a lipase, wherein the lipase is preferably lipase B from Candida antartica.

11. A reaction system for a catalyst, wherein the reaction system comprises a substrate and a catalyst, wherein the substrate is 2-bromobutyric acid, methyl 2-bromobutyrate or ethyl 2-bromobutyrate, and the substrate is preferably one or more of racemate, R-form or S-form enriched enantiomer; wherein,
when the substrate is 2-bromobutyric acid, the catalyst comprises the fluoroacetate dehalogenase mutant of claim 1; preferably, the 2-bromobutyric acid has a concentration of 0.02 g/ml-0.2 g/ml, preferably 0.1-0.15 g/ml, more preferably 0.1 g/ml; and/or, the fluoroacetate dehalogenase or mutant thereof has a concentration of 2-20 U/ml, preferably 10 U/ml.
more preferably, the reaction system further comprises a reaction solvent, wherein the reaction solvent is preferably water; and/or, pH of the reaction system is 6-8, preferably 7.

12. (canceled)

13. A method for preparing (R)-2-bromobutyric acid and/or (R)-2-hydroxybutyric acid by use of the fluoroacetate dehalogenase mutant of claim 1.

14. A preparation method of (R)-2-bromobutyric acid or (R)-2-hydroxybutyric acid, wherein the preparation method comprises following steps: a catalytic reaction is performed in the presence of the reaction system of claim 11 to obtain (R)-2-bromobutyric acid or (R)-2-hydroxybutyric acid, respectively; wherein the catalytic reaction has a reaction system with a temperature of 20-30° C., preferably 30° C.; and/or, the catalytic reaction has a reaction time of 5-10 hours, preferably 8 hours; wherein the catalytic reaction comprises a dehalogenation reaction, or a dehalogenation reaction and an ester hydrolysis reaction.

15. (canceled)

16. A catalyst combination, wherein the catalyst combination comprises (a) the dehalogenase combination of claim 9 catalyzing a halogenated substrate, and (b) a lipase, wherein the lipase is preferably lipase B from Candida antartica.

17. A reaction system for a catalyst, wherein the reaction system comprises a substrate and a catalyst, wherein the substrate is 2-bromobutyric acid, methyl 2-bromobutyrate or ethyl 2-bromobutyrate, and the substrate is preferably one or more of racemate, R-form or S-form enriched enantiomer; wherein,
when the substrate is 2-bromobutyric acid, the catalyst comprises the dehalogenase combination of claim 9 catalyzing a halogenated substrate; preferably, the 2-bromobutyric acid has a concentration of 0.02 g/ml-0.2 g/ml, preferably 0.1-0.15 g/ml, more preferably 0.1 g/ml; and/or, the fluoroacetate dehalogenase or mutant thereof has a concentration of 2-20 U/ml, preferably 10 U/ml;
more preferably, wherein the reaction system further comprises a reaction solvent, wherein the reaction solvent is preferably water; and/or, pH of the reaction system is 6-8, preferably 7.

18. A reaction system for a catalyst, wherein the reaction system comprises a substrate and a catalyst, wherein the substrate is 2-bromobutyric acid, methyl 2-bromobutyrate or ethyl 2-bromobutyrate, and the substrate is preferably one or more of racemate, R-form or S-form enriched enantiomer; wherein,
when the substrate is methyl 2-bromobutyrate or ethyl 2-bromobutyrate, the catalyst comprises the catalyst combination of claim 10; preferably, the methyl 2-bromobutyrate or ethyl 2-bromobutyrate has concentration of 0.02 g/ml-0.2 g/ml, preferably 0.1-0.15 g/ml, more preferably 0.15 g/ml; and/or, the fluoroacetate dehalogenase or mutant thereof has a concentration of 2-20 U/ml, preferably 10 U/ml; and/or, the lipase has a concentration of 1-20 g/L, preferably 10 g/L;
more preferably, the reaction system further comprises methanol, wherein a volume ratio of the methanol to the reaction system is preferably 1-10%, more preferably 10%;
more preferably, wherein the reaction system further comprises a reaction solvent, wherein the reaction solvent is preferably water; and/or, pH of the reaction system is 6-8, preferably 7.

19. A reaction system for a catalyst, wherein the reaction system comprises a substrate and a catalyst, wherein the substrate is 2-bromobutyric acid, methyl 2-bromobutyrate or ethyl 2-bromobutyrate, and the substrate is preferably one or more of racemate, R-form or S-form enriched enantiomer; wherein,
when the substrate is methyl 2-bromobutyrate or ethyl 2-bromobutyrate, the catalyst comprises the catalyst combination of claim 16; preferably, the methyl 2-bromobutyrate or ethyl 2-bromobutyrate has concentration of 0.02 g/ml-0.2 g/ml, preferably 0.1-0.15 g/ml, more preferably 0.15 g/ml; and/or, the fluoroacetate dehalogenase or mutant thereof has a concentration of 2-20 U/ml, preferably 10 U/ml; and/or, the lipase has a concentration of 1-20 g/L, preferably 10 g/L;
more preferably, the reaction system further comprises methanol, wherein a volume ratio of the methanol to the reaction system is preferably 1-10%, more preferably 10%;
more preferably, wherein the reaction system further comprises a reaction solvent, wherein the reaction solvent is preferably water; and/or, pH of the reaction system is 6-8, preferably 7.

20. A preparation method of (R)-2-bromobutyric acid or (R)-2-hydroxybutyric acid, wherein the preparation method comprises following steps: a catalytic reaction is performed in the presence of the reaction system of claim 17 to obtain (R)-2-bromobutyric acid or (R)-2-hydroxybutyric acid, respectively; wherein the catalytic reaction has a reaction system with a temperature of 20-30° C., preferably 30° C.; and/or, the catalytic reaction has a reaction time of 5-10 hours, preferably 8 hours; wherein the catalytic reaction comprises a dehalogenation reaction, or a dehalogenation reaction and an ester hydrolysis reaction.

21. A preparation method of (R)-2-bromobutyric acid or (R)-2-hydroxybutyric acid, wherein the preparation method comprises following steps: a catalytic reaction is performed in the presence of the reaction system of claim 18 to obtain (R)-2-bromobutyric acid or (R)-2-hydroxybutyric acid, respectively; wherein the catalytic reaction has a reaction system with a temperature of 20-30° C., preferably 30° C.; and/or, the catalytic reaction has a reaction time of 5-10 hours, preferably 8 hours; wherein the catalytic reaction comprises a dehalogenation reaction, or a dehalogenation reaction and an ester hydrolysis reaction.

22. A preparation method of (R)-2-bromobutyric acid or (R)-2-hydroxybutyric acid, wherein the preparation method comprises following steps: a catalytic reaction is performed in the presence of the reaction system of claim 19 to obtain (R)-2-bromobutyric acid or (R)-2-hydroxybutyric acid, respectively; wherein the catalytic reaction has a reaction system with a temperature of 20-30° C., preferably 30° C.; and/or, the catalytic reaction has a reaction time of 5-10 hours, preferably 8 hours; wherein the catalytic reaction comprises a dehalogenation reaction, or a dehalogenation reaction and an ester hydrolysis reaction.
